(19) United States
(12) Patent Application Publication
(10) Pub. No.: US 2025/0062017 A1
(43) Pub. Date: Feb. 20, 2025

(54) MEDICAL DEVICE INTEROPERABILITY METHODS, APPARATUS, AND SYSTEM

(71) Applicant: GAMBRO LUNDIA AB, Lund (SE)

(72) Inventor: Derek NEIMAN, Bethel, MN (US)

(21) Appl. No.: 18/721,129

(22) PCT Filed: Dec. 9, 2022
(86) PCT No.: PCT/EP2022/085117

§ 371 Date: Jun. 17, 2024

(30) Foreign Application Priority Data
Dec. 22, 2021 (SE) 2151584 6

(51) Int. Cl. G16H 40/60; G16H 10/60; G16H 40/20; H04W 84/12

(52) U.S. Cl. CPC G16H 40/60; G16H 10/60; G16H 40/20; H04W 84/12

(57) ABSTRACT

Medical device interoperability system, methods, and apparatus are disclosed. In an example, a server has a network map that is indicative of connectivity between access points, network switches, routers, bridges, and a plurality of medical devices that are located within a medical facility. The server receives a query message from a clinician device to identify possible other medical devices of the plurality of medical devices that are located in a same area as a first medical device. The server performs a filter routine to identify which other medical devices of the medical devices are within a close proximity of each other using the network map, displays the identified other medical devices at the clinician device, and receives a selection corresponding to a second medical device. The server then enables the second medical device to transmit medical device data to the first medical device.

### BACKGROUND

Medical networks for a healthcare facility typically connect to a significant number of medical devices. Generally, known medical networks include a local area network (“LAN”) and/or wireless LAN (“WLAN”), such as Wi-Fi or Ethernet connectivity. In such a network, connected medical devices transmit medical treatment data (e.g., sensed physiological data, treatment administration data, etc.) and/or medical event data (e.g., alarms or alerts) to a centralized gateway or server. The centralized server or gateway then stores the medical treatment data and/or medical event data to appropriate patient electronic medical records (“EMRs”). The centralized server may also transmit the medical treatment data and/or medical event data to clinician computers for monitoring. When permitted, the centralized server may also route prescriptions and/or medical device programming instructions to the appropriate medical device.

In the above-described medical network, medical devices are only configured with a destination network address of the centralized server or gateway. As such, communication is limited to connections that are between the medical devices and the centralized server or gateway. As described above, the server or gateway determines how the medical treatment data and/or medical event data is to be routed on the medical network. These known medical networks leave medical devices at edges or endpoints of the network, which limit or prevent interconnectivity of the medical devices.

Recently, the ISO/IEEE 11073TM family of standards has been created. The standards address point-of-care medical device communication by specifying an Extensible Markup Language (“XML”) Schema-based domain information models to harmonize the structure of medical information objects (e.g., structured medical treatment data and/or medical event data). The standards also define a communication model to support the exchange of medical information between medical devices using the defined medical information objects. The supported information includes medical treatment data and/or medical event data such as measurement data, settings, alerts, contextual information (e.g., patient demographics and location information), remote control, and/or archival information.

While medical device interoperability is desirable and beneficial, implementation can be problematic. For instance, many known medical systems have thousands of medical devices such as infusion pumps, dialysis machines, nutrition compounding machines, and bedside patient monitors. In addition, known medical systems have thousands of network-capable peripheral medical devices (e.g., medical sensors), such as blood pressure monitors, heart rate sensors, weight scales, pulse oxygen sensors, thermometers, electrocardiograms, glucose level sensors, etc. All of these medical devices are free to be moved between patient rooms and connected together as needed by clinicians.

As ISO/IEEE 11073TM is implemented, it is generally not desirable to enable thousands of medical devices within a medical environment to communicate with each other. Most medical devices are connected to different patients such that data sharing is not beneficial or allowed. Further, when medical devices are brought together for patient treatment, it is usually time consuming and generally inefficient for clinicians to pair these medical devices without using physical wires to connect them together. A need accordingly exists to enable clinicians to provision medical device interoperability at the network level.

### SUMMARY

Example systems, methods, and apparatus are disclosed herein that enable medical device interoperability over a medical network. The example systems, methods, and apparatus are configured to maintain a database or other data structure of networked medical devices. After receiving a request to pair a target medical device with other medical devices that are associated with a patient (e.g., located in a same room as a patient), the systems, methods, and apparatus are configured to perform a filter routine. As disclosed herein, the filter routine uses one or more rules and other criteria to determine which medical devices are physically close to each other (e.g., which medical devices are likely in a same room). The filter routine returns a list of medical devices that are likely in the same room as the target medical device. Selection of one or more of the medical devices from the list cause the systems, methods, and apparatus to transmit to the target medical device at least one of a network address or a media access control address (“MAC”) address of the selected medical device(s). The target medical device uses the received network address(es) and/or the MAC address(es) to establish a communication connection or otherwise transmit medical treatment data and/or medical event data to the selected medical device(s). Such a configuration enables only selected medical devices to communicate with each other using the ISO/IEEE 11073TM standards. The filtering routine described herein accordingly facilitates efficient medical device interoperability at the network level.

For medical devices that are connected to a common hospital network, the example systems, methods, and apparatus are configured to create a network map using a data link layer to estimate a physical layout of the entire network. The systems, methods, and apparatus apply the network map to the filter routine, which creates an algebraic expression of estimated proximity and/or distances between the medical devices. The disclosed filter routine receives the selection of the target medical device and computes estimated proximities and/or distances of the other medical devices from the target medical device. The filter routine then ranks or orders the medical devices based on their proximity to the target medical device. A closest ten, twenty, thirty, fifty, etc. medical devices are displayed. In some instances, the estimated distances are also displayed. Selection of one or more of these other medical devices causes the systems, methods, and apparatus to enable these selected medical devices to communicate with the target medical device and/or amongst each other.

In some embodiments, the filter routine may further filter the medical devices based on other criteria specified by a clinician. For example, a clinician may select to view certain types of medical devices such as infusion pumps, dialysis machines, or blood pressure sensors. Other filter criteria may include device manufacturer, data type (e.g., where filtering is performed based on the types of data output by different medical devices), treatment type (e.g., where filtering is performed based on medical devices that are needed to perform a specified treatment, such as renal replacement therapy (“RRT”), etc.

In light of the disclosure herein and without limiting the disclosure in any way, in a first aspect of the present disclosure, which may be combined with any other aspect listed herein, a control arrangement to enable medical device interoperability includes a network map of a network that is indicative of connectivity between access points, network switches, routers, bridges, and a plurality of medical devices that are located within a medical facility. Each of the medical devices includes at least one of a network address or a MAC address. The control arrangement is configured to receive a query message from a clinician device to identify possible other medical devices of the plurality of medical devices that are associated with a same patient or located in a same area as a first medical device. The first medical device is configured to use medical device data from other medical devices. The control arrangement is also configured to perform a filter routine using the network map to identify which other medical devices of the plurality of medical devices are within a same room as the patient or proximally close to each other, and cause the identified other medical devices to be displayed via a user interface of the clinician device. The control arrangement is further configured to receive a selection of one of the displayed other medical devices, the selection corresponding to a second medical device, and enable the second medical device to transmit medical device data directly to the first medical device by at least transmitting the at least one of the network address or the MAC address of the first medical device to the second medical device.

In a second aspect of the present disclosure, which may be combined with any other aspect listed herein, the control arrangement further includes a memory device configured to store the network map. In some embodiments, the network map does not provide a relation between physical locations in the medical facility and locations of the access points, the network switches, the routers, the bridges, and the plurality of medical devices.

In a third aspect of the present disclosure, which may be combined with any other aspect listed herein, the control arrangement further includes a server connected to each of the plurality of medical devices via the network, the server configured to at least perform the filter routine.

In a fourth aspect of the present disclosure, which may be combined with any other aspect listed herein, the server includes the memory device, or wherein the server is remotely located from the memory device.

In a fifth aspect of the present disclosure, which may be combined with any other aspect listed herein, at least one of the control arrangement or the server is further configured to receive a copy of the medical device data transmitted from the second medical device to the first medical device, and cause the copy of the medical device data to be displayed on the user interface of the clinician device.

In a sixth aspect of the present disclosure, which may be combined with any other aspect listed herein, the filter routine is configured to determine a physical or proximal distance between the first medical device and the plurality of medical devices by determining a network-level closest network access point, router, or bridge to the first medical device, determining network distances or proximities from the network-level closest network access point, router, or bridge, applying physical distance or proximity estimations based on the determined network distances or proximities, and selecting a number of the other medical devices that have a closest estimated physical distance or proximity to the first medical device.

In a seventh aspect of the present disclosure, which may be combined with any other aspect listed herein, the filter routine uses a data link layer to determine the network distances or proximities for creating a network map.

In an eighth aspect of the present disclosure, which may be combined with any other aspect listed herein, the server is configured to turn the network map into an algebraic expression for applying the physical distance or proximity estimations.

In a ninth aspect of the present disclosure, which may be combined with any other aspect listed herein, the number is between ten to and thirty.

In a tenth aspect of the present disclosure, which may be combined with any other aspect listed herein, the filter routine is configured to determine a physical or proximal distance between the first medical device and the plurality of medical devices by determining a network-level closest network access point, router, or bridge to the first medical device, determining network distances or proximities from the network-level closest network access point, router, or bridge, applying physical distance or proximity estimations based on the determined network distances, and selecting the other medical devices that have an estimated physical distance or proximity that is less than a threshold.

In an eleventh aspect of the present disclosure, which may be combined with any other aspect listed herein, the threshold corresponds to a size of a room or a half-room.

In a twelfth aspect of the present disclosure, which may be combined with any other aspect listed herein, the first medical device uses the medical device data from the second medical device by displaying the medical device data in conjunction with second medical device data generated by the first medical device.

In a thirteenth aspect of the present disclosure, which may be combined with any other aspect listed herein, the first medical device uses the medical device data from the second medical device by incorporating the medical device data into an equation with second medical device data generated by the first medical device to determine a medical parameter.

In a fourteenth aspect of the present disclosure, which may be combined with any other aspect listed herein, the first medical device is configured to change a treatment parameter based on the determined medical parameter.

In a fifteenth aspect of the present disclosure, which may be combined with any other aspect listed herein, the identified other medical devices are displayed in conjunction with at least one of a device identifier, a medical device type, an identifier of a medical device data type, an associated patient name, or a room location of the respective medical device.

In a sixteenth aspect of the present disclosure, which may be combined with any other aspect listed herein, each of the plurality of medical devices includes at least one of a pulse oximeter, a weight scale, a heart rate monitor, an ECG monitor, a thermometer, a pressure sensor, a hemodialysis machine, an automated peritoneal dialysis machine, a continuous renal replacement therapy (“CRRT”) machine, an infusion pump, a water purification machine, a nutrition compounding machine, a bedside monitor, or an alarm monitoring/control station.

In a seventeenth aspect of the present disclosure, which may be combined with any other aspect listed herein, at least one of the control arrangement or the server is further configured to authenticate each of the plurality of medical devices after the respective medical device is connected to the network.

In an eighteenth aspect of the present disclosure, which may be combined with any other aspect listed herein, the second medical device transmits medical device data directly to the first medical device via at least one of a Wi-Fi protocol, an Ethernet protocol, a 5G or 6G cellular protocol, a Bluetooth® wireless protocol, a Bluetooth® mesh wireless protocol, a Bluetooth® 5.0 wireless protocol, a Zigbee® wireless protocol, a Z-Wave® wireless protocol, a WeMo® wireless protocol, or a low-power wide-area network (“LPWAN”) wireless protocol.

In a nineteenth aspect of the present disclosure, which may be combined with any other aspect listed herein, the control arrangement is configured to create a record indicative that the second medical device is transmitting the medical device data directly to the first medical device.

In a twentieth aspect of the present disclosure, which may be combined with any other aspect listed herein, at least one of the control arrangement or the server is configured to cause the second medical device to stop transmitting the medical device data to the first medical device after receiving at least one of (i) an indication of a disconnection of at least one of the first medical device or the second medical device from the network, (ii) an end of treatment being performed by the first medical device, or (iii) a command received from the user interface indicative that the transmission should end.

In a twenty-first aspect of the present disclosure, which may be combined with any other aspect listed herein, a clinician device for specifying medical device interoperability includes a display screen, a processor in communication with the display screen, and a memory in communication with the processor. The memory stores instructions that define or specify an application, that when executed by the processor, cause the processor to transmit a query message to a server connected to a memory device storing a network map of a network that is indicative of connectivity between access points, network switches, routers, bridges, and a plurality of medical devices that are located within a medical facility, each of the medical devices including at least one of a network address or a MAC address. The query message includes a request to identify possible other medical devices of the plurality of medical devices that are associated with a same patient as a first medical device or located in a same area as the first medical device, the first medical device configured to use medical device data from other medical devices. The query message also causes the server to perform a filter routine using the network map to identify which other medical devices of the plurality of medical devices are within a same room as the patient or proximally close to each other. The processor also receives a message from the server that is indicative of the identified other medical devices, causes the identified other medical devices to be displayed via a user interface on the display screen, receives a selection of one of the displayed other medical devices, the selection corresponding to a second medical device, and transmits a message to the server indicative of the selection, causing the server to enable the second medical device to transmit medical device data directly to the first medical device by at least transmitting the at least one of the network address or the MAC address of the first medical device to the second medical device.

In a twenty-second aspect of the present disclosure, which may be combined with any other aspect listed herein, the display screen, the processor, and the memory are included within a smartphone, a tablet computer, a laptop computer, a desktop computer, a workstation, or a clinician station.

In a twenty-third aspect of the present disclosure, which may be combined with any other aspect listed herein, the memory stores additional instructions that define or specify the application, that when executed by the processor, cause the processor to receive the medical device data transmitted by the second medical device to the first medical device, and display the received medical device data within the same user interface or a different user interface on the display screen.

In a twenty-fourth aspect of the present disclosure, which may be combined with any other aspect listed herein, the first medical device uses the medical device data from the second medical device by displaying the medical device data in conjunction with second medical device data generated by the first medical device.

In a twenty-fifth aspect of the present disclosure, which may be combined with any other aspect listed herein, the first medical device uses the medical device data from the second medical device by incorporating the medical device data into an equation with second medical device data generated by the first medical device to determine a medical parameter.

In a twenty-sixth aspect of the present disclosure, which may be combined with any other aspect listed herein, the first medical device is configured to change a treatment parameter based on the determined medical parameter.

In a twenty-seventh aspect of the present disclosure, which may be combined with any other aspect listed herein, the identified other medical devices are displayed in conjunction with at least one of a device identifier, a medical device type, an identifier of a medical device data type, an associated patient name, or a room location of the respective medical device.

In a twenty-eighth aspect of the present disclosure, which may be combined with any other aspect listed herein, each of the plurality of medical devices includes at least one of a pulse oximeter, a weight scale, a heart rate monitor, an ECG monitor, a thermometer, a pressure sensor, a hemodialysis machine, an automated peritoneal dialysis machine, a continuous renal replacement therapy (“CRRT”) machine, an infusion pump, a water purification machine, a nutrition compounding machine, a bedside monitor, or an alarm monitoring/control station.

In a twenty-ninth aspect of the present disclosure, which may be combined with any other aspect listed herein, a system comprises the control arrangement and the clinician device according to any one of the above-aspects.

In a thirtieth aspect of the present disclosure, which may be combined with any other aspect listed herein, a method to enable medical device interoperability includes storing in a memory device a network map of a network that is indicative of connectivity between access points, network switches, routers, bridges, and a plurality of medical devices that are located within a medical facility, each of the medical devices including at least one of a network address or a MAC address. The method also includes receiving, in a server that is connected to each of the plurality of medical devices via the network, a query message from a clinician device to identify possible other medical devices of the plurality of medical devices that are associated with a same patient as a first medical devices or located in a same area as the first medical device, the first medical device configured to use medical device data from other medical devices. The method further includes performing, via the server, a filter routine using the network map to identify which other medical devices of the plurality of medical devices are within a close proximity of each other, causing, via the server, the identified other medical devices to be displayed via a user interface of the clinician device, receiving, in the server, a selection of one of the displayed other medical devices, the selection corresponding to a second medical device, and enabling, via the server, the second medical device to transmit medical device data directly to the first medical device by at least transmitting the at least one of the network address or the MAC address of the first medical device to the second medical device.

In a thirty-first aspect of the present disclosure, which may be combined with any other aspect listed herein, a method performed by a clinician device for specifying medical device interoperability includes transmitting a query message to a server connected to a memory device storing a network map of a network that is indicative of connectivity between access points, network switches, routers, bridges, and a plurality of medical devices that are located within a medical facility, each of the medical devices including at least one of a network address or a MAC address. The query message includes a request to identify possible other medical devices of the plurality of medical devices that are associated with a same patient as a first medical device or located in a same area as the first medical device, the first medical device configured to use medical device data from other medical devices. The query method causes the server to perform a filter routine using the network map to identify which other medical devices of the plurality of medical devices are within a same room as the patient or proximally close to each other. The method also includes receiving a message from the server that is indicative of the identified other medical devices, causing the identified other medical devices to be displayed via a user interface on a display screen, receiving a selection of one of the displayed other medical devices, the selection corresponding to a second medical device, and transmitting a message to the server indicative of the selection, causing the server to enable the second medical device to transmit medical device data directly to the first medical device by at least transmitting the at least one of the network address or the MAC address of the first medical device to the second medical device.

In a thirty-second aspect of the present disclosure, which may be combined with any other aspect listed herein, the method further includes receiving the medical device data transmitted by the second medical device to the first medical device, and displaying the received medical device data within the same user interface or a different user interface on the display screen.

In a thirty-third aspect of the present disclosure, any of the structure and functionality disclosed in connection with FIGS. 1 to 11 may be combined with any of the other structure and functionality disclosed in connection with FIGS. 1 to 11.

In light of the present disclosure and the above aspects, it is therefore an advantage of the present disclosure to provide efficient medical device interoperability.

It is another advantage of the present disclosure to provide an efficient computational routine to filter medical devices based on estimated proximity to a designated medical device to enable selection for interoperability.

It is further advantage of the present disclosure to create a network map of medical devices using a data link layer.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 shows a diagram of a medical network system, according to an example embodiment of the present disclosure.

FIG. 2 is a diagram of an alternative embodiment of the medical network system of FIG. 1, according to an example embodiment of the present disclosure.

FIG. 3 is a flow diagram of an example procedure for providing efficient interoperability between designated medical devices of the medical network system of FIGS. 1 and 2, according to an example embodiment of the present disclosure.

FIG. 4 is an example network map created by a server, according to an example embodiment of the present disclosure.

FIG. 5 is a diagram of a user interface of a clinician device showing closest medical devices for interoperability linking, according to an example embodiment of the present disclosure.

FIG. 6 is a message flow diagram showing a configuration procedure to enable medical device interoperability, according to an example embodiment of the present disclosure.

FIG. 7 is a diagram that is illustrative of how medical devices can be configured for interoperability, according to an example embodiment of the present disclosure.

FIG. 8 shows a user interface of a clinician device for pairing medical devices, according to an example embodiment of the present disclosure.

FIG. 9 shows a diagram that is illustrative of inter-medical device communication, according to an example embodiment of the present disclosure.

FIG. 10 is a diagram that illustrated the medical device data exchanged between medical devices, according to an example embodiment of the present disclosure.

FIG. 11 is a flow diagram of an example procedure for using a clinician device to provide interoperability between designated medical devices of the medical network system of FIGS. 1 and 2, according to an example embodiment of the present disclosure.

### DETAILED DESCRIPTION

Methods, systems, and apparatus are disclosed for providing medical device interoperability. The methods, systems, and apparatus disclosed herein are configured to create a network-level map at the data link layer that provides an estimation of medical device locations within a given medical network or hospital system. The map provides a path of connections between medical devices using known connectivity between networking equipment, such as routers, switches, gateways, access points, etc. The methods, systems, and apparatus use a filter routine that uses the network map to convert the known network locations of medical devices into algebraic expressions that estimate physical distances between all of the medical devices within the network. The physical distances between medical devices are used by the methods, systems, and apparatus to enable medical device interoperability per the ISO/IEEE 11073TM standards or other communication interoperability standards.

The methods, systems, and apparatus enable a clinician to select which medical devices are to communicate with each other, or at least specify which medical device is to receive data (or other information objects) from another medical device. Instead of allowing all medical devices to communicate with each, and instead of presenting a list of thousands of medical devices within a network, the example methods, systems, and apparatus are configured to receive a query message from a clinician that identifies a medical device, as a target or first medical device. Based on this identification, the methods, systems, and apparatus use the filter routine to identify which medical devices are estimated to be in closest proximity to the target or first medical device based on known network connectivity information. This filtering provides a list or other graphical representation that enables a clinician to quickly know which medical devices are likely in the same room as a patient. A clinician may select one or more of the displayed medical devices, which causes the methods, systems, and apparatus to transmit one or more messages to the target/first medical device and/or the selected medical device(s) with the appropriate network address, MAC address, and/or authentication information to enable device-to-device direct communication.

Reference is made herein to interoperability between medical devices. As disclosed herein, the methods, systems, and apparatus enable interoperability by providing medical devices with addressing information of other medical devices. This addressing information enables the medical devices to transmit messages to other medical devices with medical device data and/or treatment data. The messages may be communicated directly among the medical devices using network switches, routers, access points, etc. In some embodiments, the communications use hospital networking equipment, but bypass centralized servers. In other embodiments, the medical devices may communicate separate from a hospital network using a different communication protocol, such as Bluetooth®, Bluetooth® mesh, Bluetooth® low energy, Bluetooth® 5.0, Zigbee®, Z-Wave®, WcMo®, 5G/6G cellular protocols, and/or LoRa (a low-power wide-area network (“LPWAN”) network protocol). In these embodiments, the addressing information may include protocol-specific information to enable pairing with certain proximal medical devices.

Interoperability between medical devices is beneficial since it enables medical devices to share information that may be beneficial for treating a patient. For example, an infusion pump may have certain logic that stops or pauses an infusion therapy, or changes how an infusion therapy is administered, based on certain physiological conditions, such as blood pressure or heart rate. Normally, this information is entered manually by a clinician periodically, or never entered at all. With device interoperability, blood pressure, heart rate, and other physiological data may be transmitted directed to the infusion pump from peripheral sensors connected to the patient. Such a configuration provides real-time or near real-time data to an infusion pump, which enables real-time patient monitoring and treatment adjustments, thereby improving patient outcomes and treatment effectiveness.

Reference is made herein to medical device data. As disclosed, medical device data is generated at a medical device and is available for transmission. The medical device data includes treatment programming information. Treatment programming information includes one or more parameters that define how a medical device is to operate to administer a treatment to a patient. For a peritoneal dialysis therapy, the parameters may specify an amount (or rate) of fresh dialysis fluid to be pumped into a peritoneal cavity of a patient, an amount of time the fluid is to remain in the patient's peritoneal cavity (i.e., a dwell time), and an amount (or rate) of used dialysis fluid and ultrafiltration (“UF”) that is to be pumped or drained from the patient after the dwell period expires. For a treatment with multiple cycles, the parameters may specify the fill, dwell, and drain amounts for each cycle and the total number of cycles to be performed during the course of a treatment (where one treatment is provided per day or separate treatments are provided during the daytime and during nighttime). For CRRT, the parameter may include a continuous UF rate. In addition, the parameters may specify dates/times/days (e.g., a schedule) in which treatments are to be administered by the medical fluid delivery machine. Further, parameters of a prescribed therapy may specify a total volume of dialysis fluid to be administered for each treatment in addition to a concentration level of the dialysis fluid, such as a dextrose level. For an infusion therapy, the parameters may include a volume to be infused, a medication to be infused, a medication concentration, a medication dosage, and/or an infusion rate.

For the AK 98TM hemodialysis machine manufactured by Baxter International Inc., the treatment programming parameters may include a UF volume, a treatment time, a concentrate type, a sodium concentration, a bicarbonate concentration, a heparin bolus volume, a heparin flow rate, a heparin stop time, a heart rate monitoring flag, a dialysis fluid temperate, a dialysis fluid flow rate, and indications as to whether UF is isolated, UF profiling is to occur, a single needle is being used for arterial and venous connections, whether sodium profiling is to occur, whether bicarbonate profiling is to occur, and/or whether dialysis fluid conductivity monitoring is to occur. A blood flow rate to a dialyzer may also be a treatment programming parameter. For HDF, pre-and post-infusion volumes may also be treatment programming parameters.

The medical device data also includes event information that relates to administration of the treatment. The event information may include data generated by a medical device that is indicative of measured, detected, or determined parameter values. For example, while a prescribed therapy may specify that a treatment is to comprise five separate cycles, each with a 45 minute dwell time, a medical fluid delivery device may administer a treatment where fewer cycles are provided, each with a 30 minute dwell time. The medical device monitors how the treatment is administered and accordingly provides parameters that are indicative of the operation. The parameters for the treatment data may include, for example, a total amount of dialysis fluid administered to the patient, a number of cycles operated, a fill amount per cycle, a dwell time per cycle, a drain time/amount per cycle, an estimated amount of UF removed, a treatment start time/date, and/or a treatment end time/date. The treatment data may also include calculated parameters, such as a fill rate and a drain rate, determined by dividing the amount of fluid pumped by the time spent pumping. The treatment/event data may further include an identification of an alarm that occurred during a treatment, a duration of the alarm, a time of the alarm, an event associated with the alarm, and/or an indication as to whether the issue that caused the alarm was resolved or whether the alarm was silenced.

The medical device data further includes device machine logs that include diagnostic information, fault information, etc. The diagnostic information may include information indicative of internal operations of a medical device, such as faults related to pump operation, signal errors, communication errors, software issues, etc. The medical device data may be transmitted as a data stream or provided at periodic intervals. In some instances, the medical device data may be transmitted as events or other changes to the data occur.

### I. Medical Network System Embodiment

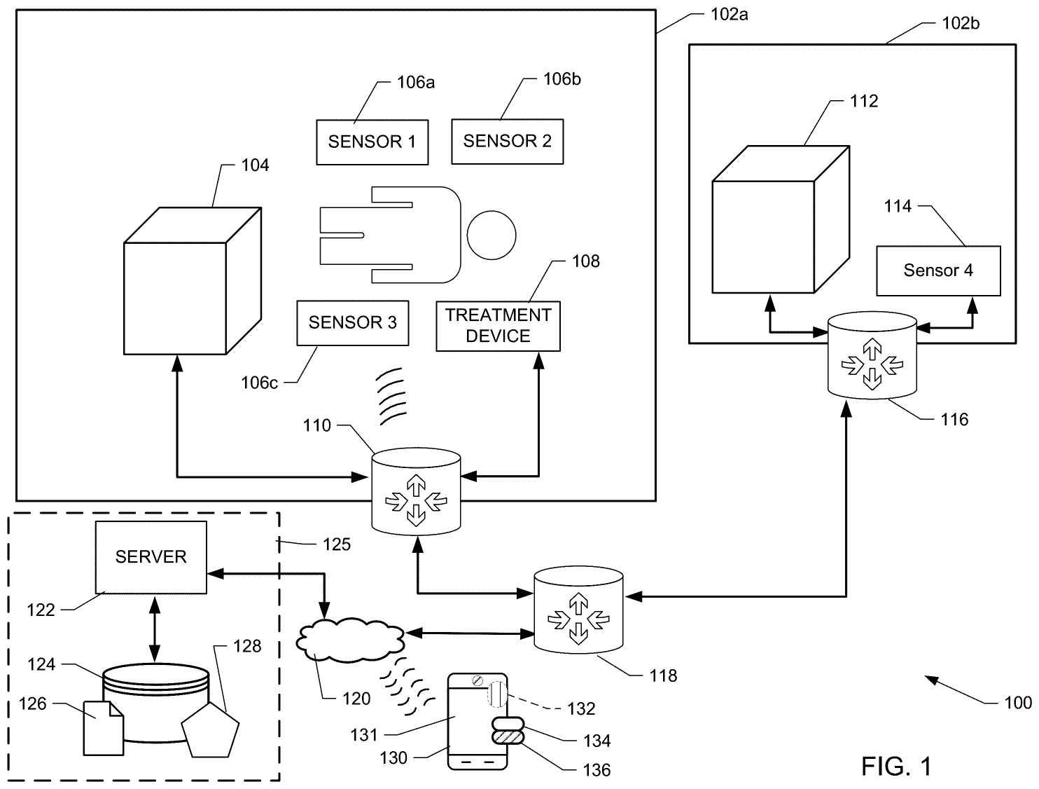

FIG. 1 shows a diagram of a medical network system 100, according to an example embodiment of the present disclosure. The example medical network system 100 includes a first patient room 102a and a second patient room 102b. The patient rooms 102 may be part of a medical facility, such as a clinic or hospital. The system 100 may include additional patient rooms. For instance, the system 100 may include tens, hundreds, or thousands of patient rooms that may be located in a single building or spread out over multiple buildings.

The first patient room 102a includes a dialysis machine 104, physiological sensors 106, and a treatment device 108, which may collectively represent medical devices. In the illustrated example, the dialysis machine 104 may be the PrisMax CRRT machine manufactured by Baxter International Inc. It should be appreciated that in other embodiments, the dialysis machine 104 may include any other renal failure therapy machine, such as an intermediate hemodialysis machine or a peritoneal dialysis machine. The treatment device 108 includes another medical device, such as an infusion pump (e.g., a syringe pump, a linear peristaltic pump, a large volume pump (“LVP”), an ambulatory pump, multi-channel pump), a nutritional compounding machine, a water preparation machine, etc. The treatment device 108 may also include a bedside patient monitor. The physiological sensors 106 each includes an oxygen sensor, a respiratory monitor, a glucose meter, a blood pressure monitor, an electrocardiogram (“ECG”) monitor, a weight scale, a heart rate monitor, or any other peripheral medical device configured to sense a physiological parameter of a patient.

The example dialysis machine 104 is configured to accept one or more parameters specifying a treatment or prescription (i.e., treatment programming information). During operation, the dialysis machine 104 generates event, diagnostic, and/or operational data (e.g., medical device data). Further, the treatment device 108 may also accept one or more parameters specifying a treatment or prescription. The treatment device 108 may also generate medical device data. The example sensors further 106 generate medical device data that includes patient physiological data. In some embodiments, the medical device data conforms to the ISO/IEEE 11073TM standards as XML-based information objects. In other embodiments, the medical device data is in a different format, such as JavaScript Object Notation (“JSON”), a HyperText Markup Language (“HTML”), a comma-separated values (“CSV”), text, and/or Health-Level-7 (“HL7”).

The example dialysis machine 104 and/or the treatment device 108 may include one or more control interfaces for displaying instructions and receiving control inputs from a user. The control interface may include buttons, a control panel, or a touchscreen. The control interface may also be configured to enable a user to navigate to a certain window or user interface on a screen of the dialysis machine 104 and/or the treatment device 108. The control interface may also provide instructions for operating or controlling the dialysis machine 104 and/or the treatment device 108.

The example dialysis machine 104 and/or the treatment device 108 also includes a processor. The processor of the dialysis machine 104 and/or the treatment device 108 operates according to one or more instructions for performing a treatment on a patient. The instructions may be acquired via the control interface. The processor also monitors devices components for issues, which are documented as diagnostic medical device data. The processor creates medical device data in conjunction with operating one or more pumps or other components to administer the treatment. The processor further transmits the medical device data.

In the illustrated example of FIG. 1, the dialysis machine 104, the physiological sensors 106, and the treatment device 108 are communicatively coupled to a network switch 110.

The dialysis machine 104, the physiological sensors 106, and the treatment device 108 may be communicatively coupled to the network switch 110 via a wired connection such as an Ethernet or USB connection, or a wireless connection such as a Wi-Fi connection, a wireless local area network (“WLAN”), and/or a cellular 5G/6G connection. The network switch 110 may include one or more of an access point, a router, repeater, or other telecommunication equipment for routing communications in a network. In some embodiments, the network switch 110 includes a routing-and-forwarding table that associates local addresses, network addresses, and/or MAC addresses with each of the dialysis machine 104, the physiological sensors 106, and the treatment device 108. In some instances, the server 122 is configured to authenticate each of the plurality of medical devices 104, 106, and 108 after the respective medical device is connected to the network 120.

Similar to the first patient room 102a, the second patient room 102b includes a dialysis machine 112 and a physiological sensor 114. The second patient room 102b also includes a network switch 112. In some embodiments, the patient rooms 102 may include additional network switches or other networking equipment.

Each of the network switches 110 and 116 are communicatively coupled to another network switch 118, which is communicatively coupled more generally to a medical network 120. As such, each of the network switches 110, 116, and 118 may be part of the medical network 120 in addition to other routers, access points, switchers, repeaters, gateways, hubs, network appliances, etc. that are not shown for brevity. Each of the switches and routers includes a routing-and-forwarding table that identifies a next switch/router/device for transmitting messages through the network 120 using an Internet Protocol, cellular protocol, or combinations thereof.

The example medical network 120 is also communicatively coupled to a server 122, which may include any processor, laptop computer, desktop computer, tablet computer, workstation, control logic, etc. provisioned in a singular or distributed computing environment. While a single server 122 is shown, the medical network system 100 may include additional servers. The servers may provide medical system management and include a service portal, an enterprise resource planning system, a web portal, a business intelligence portal, a HIPAA compliant database, a pharmacy system, etc.

The server 122 is communicatively coupled to a memory device 124. In some embodiments, the connection may be through the network 120. Together, the server 122 and the memory device 124 may form a control arrangement 125 to enable medical device interoperability. The memory device 124 may be integrated with the server 122 or provided separately from the server 122.

The memory device 124 is configured to store medical device data 126 that is generated and/or transmitted by the medical devices 104, 106, 108, 112, and 114 to the server 122. The memory device 124 may store individual patient records, known as electronic medical records (“EMRs”). The server 122 may use a patient identifier provided in conjunction with the medical device data 126 to determine which patient EMR is to receive the data. The memory device 124 may store the medical device data 126 in a HL7 format, an XML format, a binary version 2 format, a binary version 3 format, or a Fast Healthcare Interoperability Resources (“FHIR”) format. The memory device 124 may include any RAM, ROM, EEPROM, flash drive, solid state drive, distributed database, etc.

The memory device 124 is also configured to store a filter routine 128. The server 122 is configured to execute one or more instructions specified by the filter routine 128 to perform the operations discussed here. For example, the filter routine 128 is configured to create a network mapping of the medical devices 104, 106, 108, 112, and 114 at the data link layer based on known locations of the network switches 110, 116, and 118 in addition to other network equipment of the network 120. The locations may be physical, such as specifying certain rooms or other indoor locations. Alternatively, the locations may be logical based on communicative connections between the medical devices 104, 106, 108, 112, and 114, the network switches 110, 116, and 118 and/or the network 120. As described below, the filter routine 128 uses the network map to create algebraic expressions or other logical relations to determine relative distances and/or proximities between the medical devices 104, 106, 108, 112. The algebraic expressions or other logical relations enable the filter routine 128 to determine which of the medical devices 104, 106, 108, 112, and 114 are most closely located to a specified or target (first) medical device within the medical network system 100.

FIG. 1 also shows that the medical network system 100 includes a clinician device 130. The example clinician device 130 includes an application 132 that is configured to interface with the server 122 for at least configuring interoperability among specified medical devices. The application 132 may include one or more user interfaces with data fields that are configured to receive a query message or other input to select medical devices for interoperability. The application 132 may also provide one or more user interfaces showing a graphical representation of the network map and/or a list of closely located medical devices in relation to a target (first) medical device. The application 132 may further include inputs for enabling medical devices to communicate directly with each other and/or for displaying medical device data 126 received from the medical devices and/or a copy of medical device data 126 communicated between medical devices. The application 132 may additionally include interfaces for remotely programing or otherwise controlling at least some of the medical devices.

The clinician device 130 also includes a processor 134 that is in communication with a display screen 131 and 1 memory 136 storing instructions. At least some of the instructions define or specify the application 132, that when executed by the processor 134, cause the processor 134 to provide interfaces for performing the operations described herein. The processor 134 may comprise digital and/or analog circuitry structured as a microprocessor, application specific integrated circuit (“ASIC”), controller, etc. The memory 136 includes a volatile or non-volatile storage medium. Further, the memory 136 may include any solid state or disk storage medium.

The clinician device 130 may include a smartphone, tablet computer, laptop computer, desktop computer, workstation, clinician station, etc. While FIG. 1 shows a single clinician device 130, it should be appreciated that the medical network system 100 may include a plurality of clinician devices 130 that are connected to the server 122 via the network 120. Further, the network 120 may include gateways and/or firewalls to enable external clinical devices 130 to communicate with the server 122.

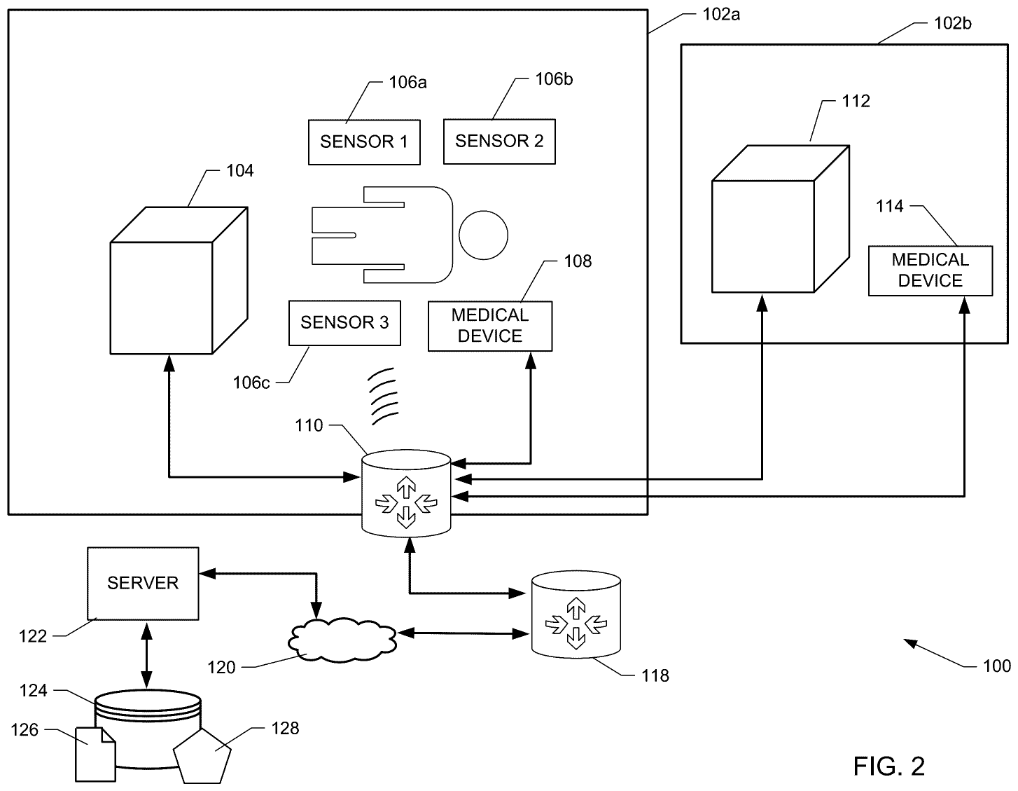

FIG. 2 is a diagram of an alternative embodiment of the medical network system 100 of FIG. 1, according to an example embodiment of the present disclosure. In this embodiment, the medical devices 112 and 114 of the second patient room 102b are communicatively coupled to the network switch 110. Such a configuration shows that there is not necessarily a one-to-one correspondence between patient rooms and a local router or patient switch. Medical networks are often complex and varied, and oftentimes have medical devices from different rooms coupled to a common switch or router. The filter routine 128 may be configured to use port numbers or other designations of the network switch 110 to determine, for example, relative locations of the medical devices between the patient rooms 102. It should be appreciated that there is a virtually limitless number of configurations of medical devices and networking equipment within the medical network system 100.

### II. Example Interoperability Configuration Embodiment

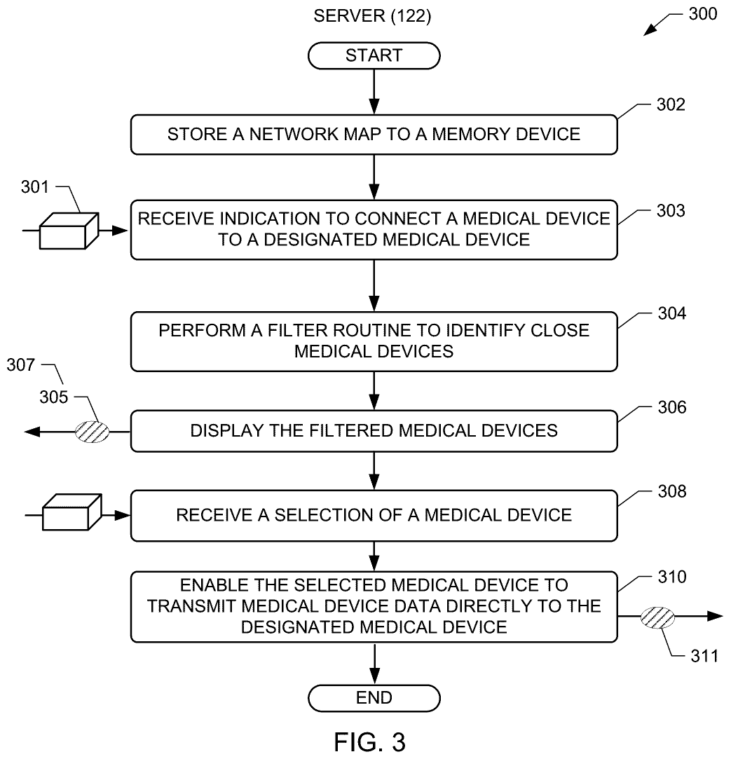

FIG. 3 is a flow diagram of an example procedure 300 for providing efficient interoperability between designated medical devices of the medical network system 100 of FIGS. 1 and 2, according to an example embodiment of the present disclosure. Although the procedure 300 is described with reference to the flow diagram illustrated in FIG. 3, it should be appreciated that many other methods of performing the steps associated with the procedure 300 may be used. For example, the order of many of the blocks may be changed, certain blocks may be combined with other blocks, and many of the blocks described may be optional. For example, enabling medical devices to communicate with each other may include transmitting messages to only one of the medical devices, all of the designed medical devices, or specified medical devices that are to transmit medical device data to other target medical devices of the medical network system 100. The operations described in the procedure 300 are specified by one or more instructions and may be performed among multiple devices including, for example, the server 122, the clinician device 130, and/or the medical devices.

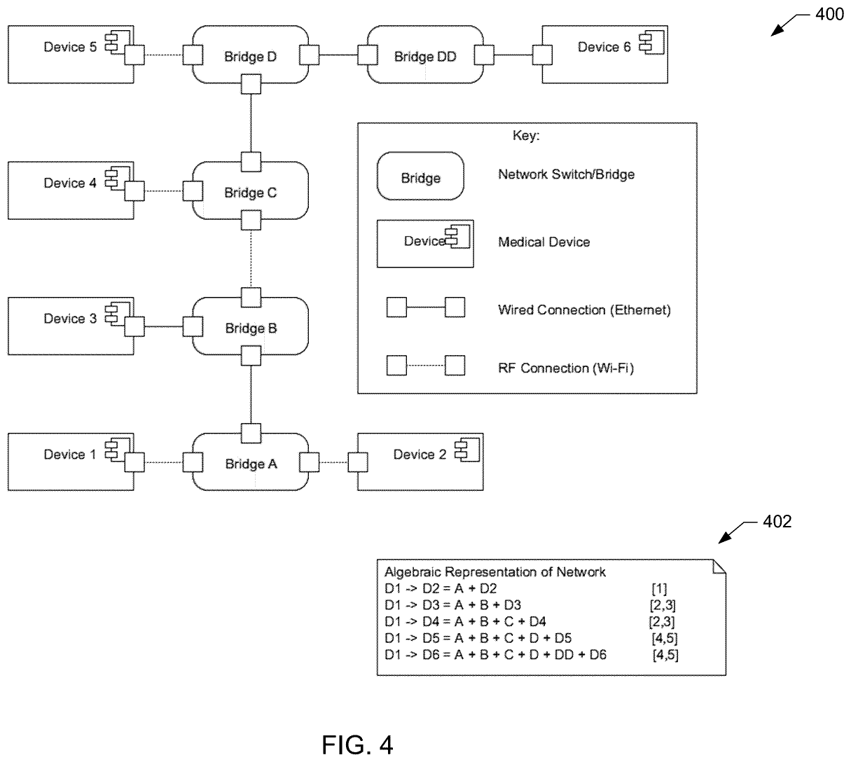

The example procedure 300 may be performed after the server 122 has already created a network mapping and algebraic expressions to determine relative and/or absolute distances between medical devices of the medical network system. FIG. 4 is an example network map 400 created by the server 122, according to an example embodiment of the present disclosure. The network map 400 includes relative positions and connections (e.g., connectivity) between medical devices (i.e., Devices 1 to 6) and network switches/bridges (i.e., Bridges A to DD). The network map 400 also identifies wired and wireless connections. In some embodiments, the network map 400 may also identify port numbers, device network addresses, MAC addresses, and/or device identifiers. In some embodiments, the network map 400 does not provide a relation between physical locations in a medical facility and locations of the access points, the network switches, the routers, the bridges, and the plurality of medical devices because such information may not be available or easily obtainable.

FIG. 4 also shows an example algebraic representation 402 of the network map 400.

The algebraic representation 402 may be created by the server 122 executing the filter routine 128. In an example, the components in the medical network system 100 report a unique MAC address and the device's supported system capabilities. Each unique MAC address representing a device is represented as a variable (e.g., A, B, C . . . ) in a plurality of equations. The distance or proximity estimated between two endpoint medical devices is represented by an equation. For this example, all distances or proximities are referenced from the perspective of Device 1 (e.g., a target or first device), but the same process holds true for each individual medical device. The distance or proximity between Device 1 and Device 2 is represented by the variable D12, and so on. Solving for all endpoints on the network causes the server 122 to generate the following equations for the filter routine 128:

$${D}_{12} = A + {D}_{2} \\ {D}_{13} = A + B + {D}_{3} \\ {D}_{14} = A + B + C + {D}_{4} \\ {D}_{15} = A + B + C + D + {D}_{5} \\ {D}_{16} = A + B + C + D + DD + {D}_{6}$$

It can be assumed that the network layout or connectivity between rooms, devices, switches, etc. should all be similar. As such, the server 122 approximates that the end point medical devices are approximately the same distance or proximity from their closest bridge or switch. Additionally, the server 122 is configured to assume that all distances or proximities between medical devices are some number greater than 0. For example, the server 122 may be configured to assume:

$${D}_{1} \underline{\approx } {D}_{2} \underline{\approx } {D}_{3} \underline{\approx } {D}_{4} \underline{\approx } {D}_{5} \underline{\approx } {D}_{6} \\ A , B , C , D , DD > 0$$

Using the assumptions above, the server 122 may simply the equations as shown below for the filter routine 128, where c is some constant value:

$${D}_{12} = c \\ {D}_{13} = c + B \\ {D}_{14} = c + B + C \\ {D}_{15} = c + B + C + D \\ {D}_{16} = c + B + C + D + DD$$

Rearranging the equations in like terms the following equations are generated:

$${D}_{12} = c \\ {D}_{13} = {D}_{12} + B \\ {D}_{14} = {D}_{13} + C \\ {D}_{15} = {D}_{14} + D \\ {D}_{16} = {D}_{15} + DD$$

From the assumption that A,B,C,D,DD>0, it can be concluded by the server 122 that:

$${D}_{12} < {D}_{13} < {D}_{14} < {D}_{15} < {D}_{16}$$

The filter routine 122 may create similar expressions for each of the medical devices within the medical network system 100.

Returning to FIG. 3, the server 122 is configured to use the algebraic representation 402 of the network map 400 to perform the procedure 300. The example procedure 300 begins when the server 122 stores the network map 400 to the memory device 124 (block 302). The server 122 next receives a query message 301 from a clinician device 130 (e.g., the application 132) (block 303). The query message 301 specifies a target or first medical device. The query message 301 may also specify an instruction to identify possible other medical devices of a plurality of medical devices of the hospital network system 100 that are associated with a same patient as the first or target medical device. In the example of FIG. 1, the target/first medical device may include the dialysis machine 104, where the query message 301 requests that the server 122 identify other medical devices that are likely to also be in the first patient room 102a.

After receiving the query message 301, the server 122 is configured to use the filter routine 128 to identify medical devices that are likely to be in a same room or area as the first or target medical device (block 304). In the example above, the target (first) medical device is Device 1 such that the filter routine 128 determines a relational distance, a proximal, and/or a physical distance of the other medical devices in relation to Device 1. The relational distance, the proximal, and/or the physical distances may be calculated in part based on knowledge of the network-level connections and known distances between components of these connections or specified standard distances based on averages in network/device routing/layout. The server 122 is configured to select a certain number of closest medical devices, which may include selecting a closet ten, twenty, thirty, fifty, etc. medical devices. In other embodiments, the server 122 estimates physical distances of the medical devices using the relational positioning, separation between network devices, and/or physical placement assumptions of the network devices (e.g., network devices may be assumed to be spaced apart between 30 to 50 feet to maintain wireless signal strength (or based on standard cable lengths) in an indoor environment). In these other embodiments, the server 122 selects medical devices that are within a threshold distance, which may be ten feet, twenty fect, thirty fect, etc.

In some embodiments, the filter routine 128 is configured to determine a physical or proximal distance between the first medical device and the plurality of medical devices by determining a network-level closest network access point, router, or bridge to the first medical device and determining network distances or proximities from the network-level closest network access point, router, or bridge. The filter routine 128 may then apply physical distance or proximity estimations based on the determined network distances or proximities. The filter routine 128 next selects a number (e.g., a number between ten to and thirty) of the other medical devices that have a closest estimated physical distance or proximity to the first medical device. The filter routine 128 may use a data link layer to determine the network distances, connectivity, or proximities for creating a network map. Further, the server 122 is configured to turn the network map into an algebraic expression for applying the physical distance or proximity estimations.

In other embodiments, the filter routine 128 is configured to determine a physical or proximal distance between the first medical device and the plurality of medical devices by determining a network-level closest network access point, router, or bridge to the first medical device and determining network distances, connectivity, or proximities from the network-level closest network access point, router, or bridge. The filter routine 128 may then apply physical distance, connectivity, or proximity estimations based on the determined network distances and select the other medical devices that have an estimated physical distance, connectivity, or proximity that is less than a threshold. In some instances, the threshold corresponds to a size of a room or a half-room.

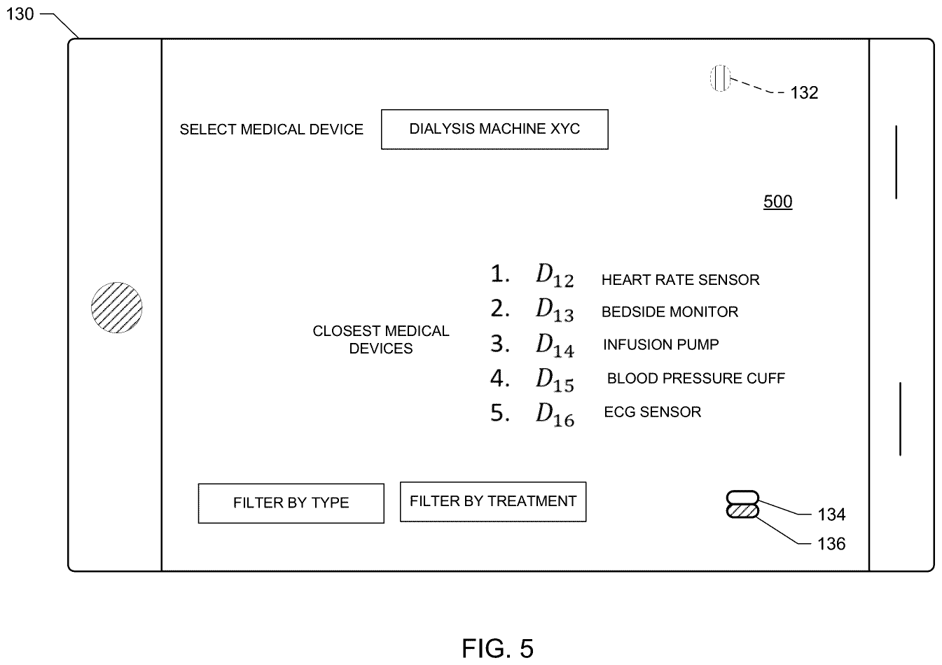

The server 122 then transmits a response message 305 to the clinician device 130 with the selected closest medical devices (block 306). FIG. 5 is a diagram of a user interface 500 of the clinician device 130 showing information from the response message 305, according to an example embodiment of the present disclosure. The user interface 500 shows that a user selected a dialysis machine XYC as the target or first medical device. An identifier of the dialysis machine XYC is included in the query message 301, which is used by the filter routine 128 of the server 122 to identify closest located medical devices. The server 122 causes these closest medical devices to be displayed in the user interface 500 as a selectable list. The list may include an identifier of the medical device and a type of medical device. In further embodiments, the list may also include an estimated distance of the medical device to the target medical device and/or an indication as to whether the medical device is transmitting data or is otherwise operational.

The example user interface 500 also may include options for other filter criteria. For example, the user interface 500 enables a user to filter medical devices by type and patient treatment. In an example, a clinician may need to link a blood pressure sensor to a dialysis machine, and thus may filter the medical devices to only view closest blood pressure sensors. In another example, the clinician may be configuring medical devices to perform a multichannel infusion, and accordingly selects a multichannel infusion treatment type to identify medical devices needed to perform such a treatment.

Returning to FIG. 3, the server 122 receives a selection message 307 of at least one of the listed medical devices (block 308). The server 122 then enables the selected medical device to transmit medical device data 126 to the target or first medical device (block 310). To enable this interoperability, the server 122 may transmit a message 311 to the selected medical device(s) with a network address and/or MAC address of the target or first medical device. The message 311 may also include an instruction to transmit medical device data to the first or target medical device. The second medical device receives the message and accordingly transmits messages directly to the first medical device using the network 120 or separate from the network.

The first medical device may incorporate the received medical device data into an equation with its own medical device data to determine a medical parameter. The first medical device may be configured to change a treatment parameter based on the determined medical parameter. Additionally or alternatively, the first medical device may display the medical device data from the second medical device with its own generated medical device data. In one embodiment, the second medical device could automatically adjust a treatment parameter of the first medical device (or vice versa) when a certain event occurs. In another embodiment, a clinician may manually adjust a parameter of the first medical device using a graphical user interface of the second medical device (or vice versa).

In some embodiments, the clinician may select only certain types of medical device data to be sent to the first or target medical device. For example, an infusion pump may report a volume of fluid infused, events, and alerts. However, a clinician may only select that the volume of fluid infused medical device data is to be transmitted to another medical device. In this instance, the user interface 500 may provide selectable options corresponding to the different types of data generated by each medical device. As such the message 311 may further identify the data type that is to be transmitted to the first or target medical device.

In some embodiments, the server 122 transmits the message 311 to the first or target medical device instead. The first or target medical device uses the MAC address and/or network address of the second medical device to transmit a request for data message, thereby enabling a pairing between devices. In yet further embodiments, the server 122 transmits messages to both the first/target medical device and the selected medical device. Further, in some instances, the user interface 500 may enable a clinician to select whether the selected medical device is to transmit data to the first/target medical device or is to receive medical device data from the target/first medical device. The message 311 accordingly provides an indication to the appropriate medical device as to what data is to be transmitted to which medical device. In yet further embodiments, the transmission of the message 311 enables the selected medical device and the target medical device to communicate bidirectional.

In instances where the medical devices communicate outside of the network, the message 311 may include protocol or pairing information. For example, the message 311 may include information to enable the medical devices to communicate directly using Bluetooth®, Bluetooth® mesh, Bluetooth® low energy, Bluetooth® 5.0, Zigbee®, Z-Wave®, WeMo®, 5G/6G cellular protocols, and/or LoRa (a low-power wide-area network (“LPWAN”) network protocol). The message 311 may also identify the protocol and/or provide authentication information, which is stored in the memory device 124 in conjunction with the network addresses and/or MAC address of each medical device of the medical network system 100.

After communication is enabled between medical devices, the example procedure 300 ends. The procedure 300 may restart when the clinician selects another medical device for interoperability. Further, the server 122 may update the algebraic representation 402 as medical devices are added or removed from the system 100, and/or as medical devices are physically moved between rooms.

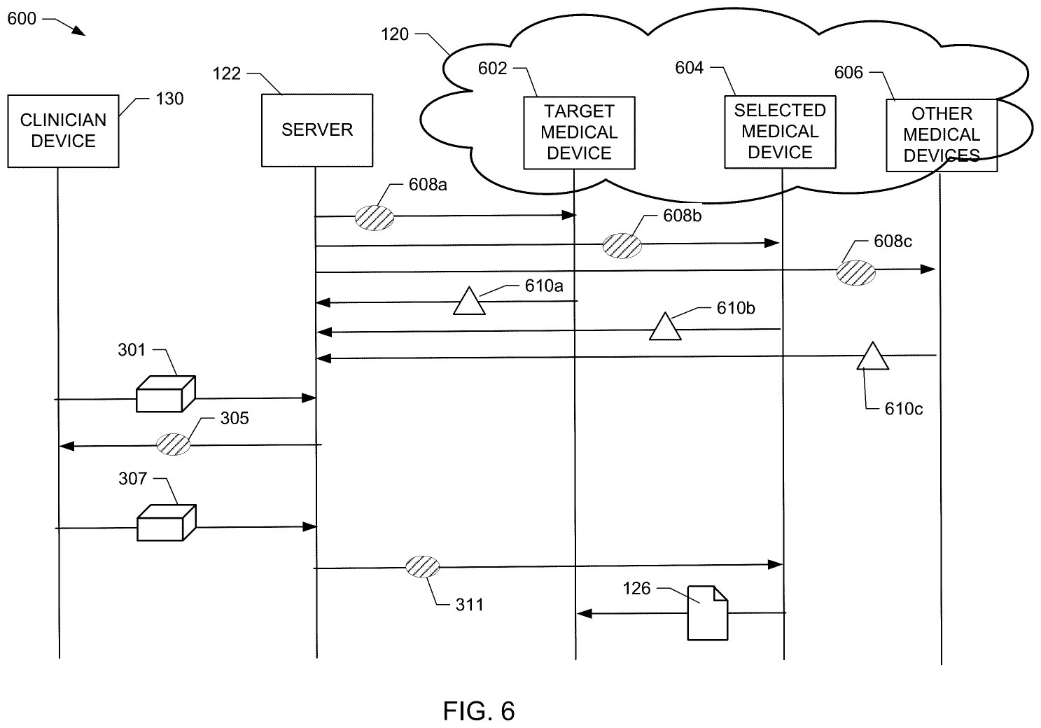

FIG. 6 is a message flow diagram showing a configuration procedure 600 to enable medical device interoperability, according to an example embodiment of the present disclosure. FIG. 6 shows that a target medical device 602, a selected medical device 604, and other medical devices 606 are part of the network 120, which is communicatively coupled to the server 122. In the illustrated example, the server 122 transmits ping messages 608a, 608b, and 608c respective to the medical devices 602, 604, and 606. In response, the medical devices 602, 604, and 606 transmit response messages 610a, 610b, 610c. The server 122 uses the messages 608 and 610 to create a network map 400 based on how those messages are routed through the network 120. The server 122 may also transmit ping messages 608 to the network components to determine connectivity within the network. The response messages 610 may also include a device identifier, network address, and/or MAC address of the respective medical device 602, 604, 606. At this point, the server 122 creates the algebraic representation of the network map for the filter routine 128.

At some time later, the clinician device 130 transmits the query message 301 to the server 122, which identifies the target medical device 602. The server 122 uses the filter routine 128 and the algebraic representations to determine the most closely located medical devices among the other medical devices 606 (including the selected medical device 604, which at this point has not yet been selected). The server 122 transmits the response message 305 to the clinician device 130 with a list of closest medical devices. The clinician device 130 then transmits the selection message 307 to the server 130, which identifies the selected medical device 604. In response, the server 122 transmits a connection message 311 to, for example, the selected medical device 604. The connection message 311 includes a network address, MAC address, authentication information, and/or protocol pairing information for the target medical device 602.

As discussed above, the connection message 311 may also identify medical device data types for transmission. After receiving the message 311, the selected medical device 604 transmits its locally generated medical device data 126 to the target medical device 602. In some embodiments, the selected medical device 604 first authenticates and/or pairs with the target medical device 602 before the data 126 can be transmitted. The selected medical device 604 may also transmit a copy of this data 126 to the server 122 (or an indication that the interoperability is enabled), which may be provided for display by the application 132 of the clinician device 130. At this point, the target medical device 602 operates using the medical device data 126 from the selected medical device 604. As can be appreciated, the example configuration procedure 600 enables a clinician to enable medical device interoperability between certain medical devices quickly and efficiently.

### III Medical Device Interoperability Embodiment

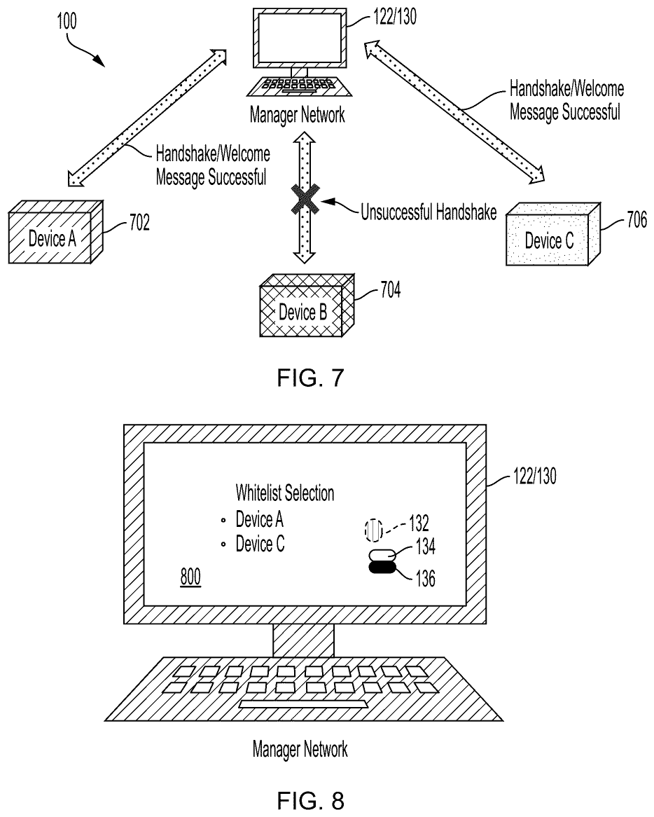

FIG. 7 is a diagram that is illustrative of how medical devices can be configured for interoperability, according to an example embodiment of the present disclosure. In this example, first medical device 702 and second medical device 706 register and/or exchange handshake messages with the server 122. This is similar to the ping message 708 and response message 710 discussed in connection with FIG. 7. This registration makes the medical devices 702 and 706 available for interoperability communication. In this example, a medical device 704 is not able to register or communicate with the server 122. As such, this medical device 704 is not made available by the server 122 for sharing medical device data.

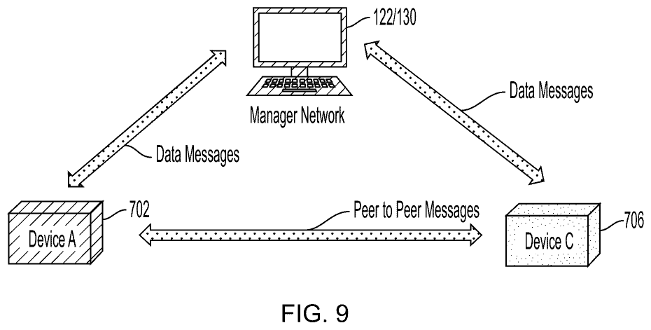

FIG. 8 shows a user interface 800 of the clinician device 130 for pairing the medical devices 702 and 706, according to an example embodiment of the present disclosure. In this example, the server 122 determines that the first medical device 702 and the second medical device 706 are close to each other using the filter routine 128 and available for sharing medical device data for treating a common patient. FIG. 9 shows a diagram that is illustrative of inter-medical device communication, according to an example embodiment of the present disclosure. As shown, the first medical device 702 and the second medical device 706 still transmit medical device data to the server 122 and/or the clinician device 130. However in addition, the first medical device 702 and the second medical device 706 transmit at least some medical device data directly between themselves without passing the data through the server 122. Such a configuration enables the medical device data to be provided more quickly between devices in a native format without having to go through formatting or other processing at the server 122.

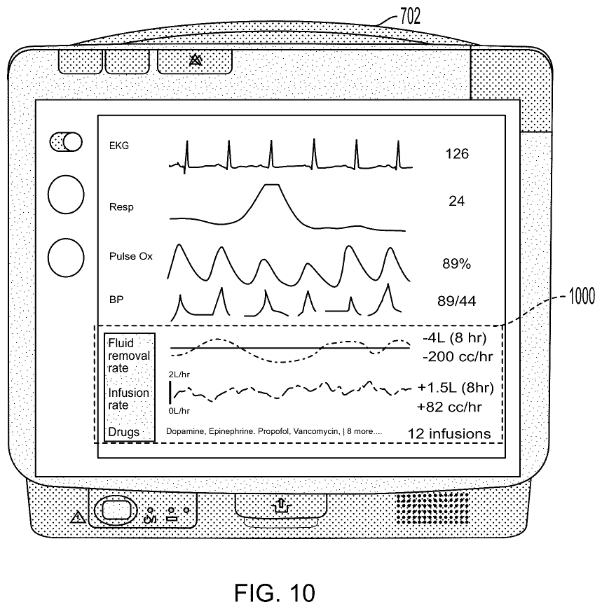

FIG. 10 is a diagram that illustrated the medical device data exchanged between medical devices, according to an example embodiment of the present disclosure. In this embodiment, the first medical device 702 is a patient bedside monitor. The first medical device 702 may include wired connections to some sensors, such as an EKG sensor, respiratory sensor, pulse oxygen sensor, and a blood pressure sensor. As such, this data is transmitted directly to the first medical device 702 via the direct wired connection. However, the second medical device 706, which may be an infusion pump, is not directly connected to the bedside monitor first medical device 702. Instead, a clinician uses the clinician device 130 and the user interface 500 provided by the application 132 to select that the bedside monitor first medical device 702 is to receive certain infusion medical device data from the infusion pump second medical device 706. The clinician may specify that a fluid removal rate, infusion rate, and drug names be transmitted to the first medical device 702. After the connection is established between the first medical device 702 and the second medical device 706 (or the second medical device 706 receives an address of the first medical device 702), the infusion pump second medical device 706 transmits the selected medical device data to the first medical device 702. As shown in FIG. 10, the received infusion medical device data is displayed within an area 1000 of the bedside monitor. Such a configuration enables the bedside monitor to provide additional medical device data to provide a more comprehensive picture of a patient's condition. Further, the bedside monitor may enable a clinician to set alarms that is based on certain combinations of the medical device data that may otherwise not be possible without the infusion medical device data. The example system, methods, and apparatus accordingly provide for efficient medical device interoperability to improve patient care.

### IV. Clinician Device Embodiment

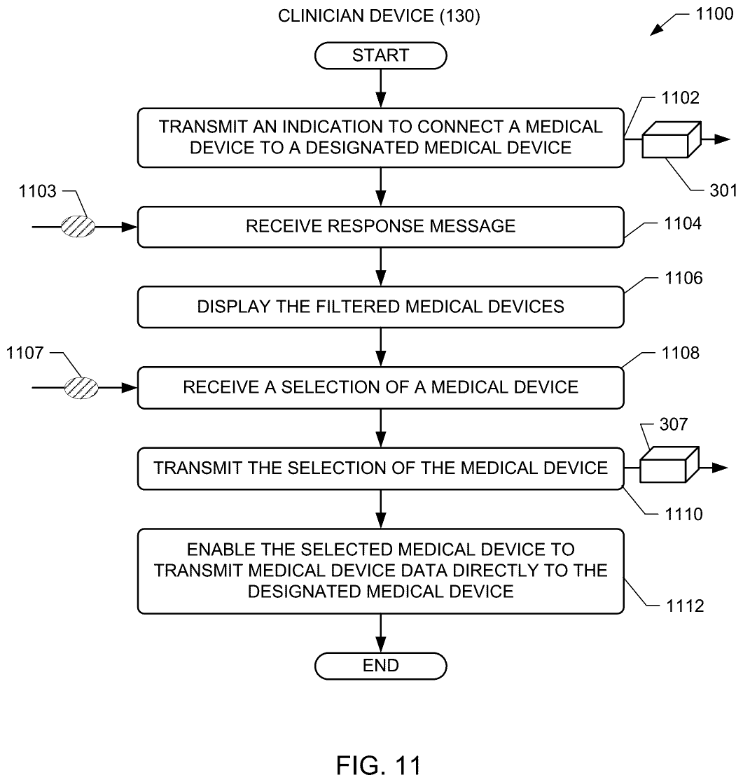

As discussed above, the clinician device 130 is configured for specifying medical device interoperability. FIG. 11 is a flow diagram of an example procedure 1100 for using the clinician device 130 to provide interoperability between designated medical devices of the medical network system 100 of FIGS. 1 and 2, according to an example embodiment of the present disclosure. Although the procedure 1100 is described with reference to the flow diagram illustrated in FIG. 11, it should be appreciated that many other methods of performing the steps associated with the procedure 1100 may be used. For example, the order of many of the blocks may be changed, certain blocks may be combined with other blocks, and many of the blocks described may be optional. For example, enabling medical devices to communicate with each other may include transmitting messages to only one of the medical devices, all of the designed medical devices, or specified medical devices that are to transmit medical device data to other target medical devices of the medical network system 100. The operations described in the procedure 1100 are specified by one or more instructions and may be performed among multiple devices including, for example, the server 122, the clinician device 130, and/or the medical devices.

The procedure 1100 begins when the clinician device 130 receives via the application 132 a request to identify possible other medical device of the plurality of medical devices that are associated with a same patient as a first medical device. The request may identify, for example, a target or first medical device. The clinician device 130 transmits a query message 301 to the server 122 including a request to identify possible other medical device of the plurality of medical devices that are associated with a same patient as a first medical device (block 1102). In this embodiment, the first medical device is configured to use medical device data from other medical devices. Further, as discussed above, the server 122 is connected to the memory device 124, which stores a network map of a network that is indicative of access points, network switches, routers, bridges, and a plurality of medical devices location within a medical facility. Each of the medical devices includes at least one of a network address or a MAC address, the query message (310). The transmission of the message 310 causes the server 122 to perform the filter routine 128 to identify which other medical devices of the plurality of medical devices are within a same room as the patient or proximally close to each other.

The clinician device 130 next receives a response message 1103 from the server 122 that is indicative of the identified other medical devices (block 1104). The clinician device 130 via the application 132 causes the identified other medical devices to be displayed via a user interface on the display screen 131 (block 1106). The clinician device 130 then receives a selection 1107, via the application 132 of one of the displayed other medical devices, the selection corresponding to a second medical device (block 1108).

The clinician device 130 then transmits a message 307 to the server 122 that is indicative of the selection of the medical device (1110). The message 307 causes the server 122 to enable the second medical device to transmit medical device data 126 directly to the first medical device by at least transmitting the at least one of the network address or the MAC address of the first medical device to the second medical device (block 1112). The procedure 1100 may then end. In some embodiments, the clinician device 130 receives and displays via the application 132 medical device data that is transmitted from the second medical device to the first medical device.

### V. CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1-52. (canceled)

53. A control arrangement to enable medical device interoperability, the control arrangement including a network map of a network that is indicative of connectivity between access points, network switches, routers, bridges, and a plurality of medical devices that are located within a medical facility, each of the plurality of medical devices including at least one of a network address or a MAC address,
wherein the control arrangement is configured to:
receive a query message from a clinician device to identify possible other medical devices of the plurality of medical devices that are associated with a same patient or located in a same area as a first medical device, the first medical device configured to use medical device data from the other medical devices,
perform a filter routine using the network map to identify which of the other medical devices of the plurality of medical devices are proximally close to each other or connected to a common router or access point,
cause the identified other medical devices to be displayed via a user interface of the clinician device,
receive a selection of one of the displayed other medical devices, the selection corresponding to a second medical device, and
enable the second medical device to transmit the medical device data to the first medical device by transmitting at least one of the network address or the MAC address of the first medical device to the second medical device.

54. The control arrangement of claim 53, further comprising a memory device configured to store the network map,
wherein the network map does not provide a relation between physical locations in the medical facility and locations of the access points, the network switches, the routers, the bridges, and the plurality of medical devices.

55. The control arrangement of claim 53, further comprising a server connected to each of the plurality of medical devices via the network, the server configured to at least perform the filter routine.

56. The control arrangement of claim 55, wherein at least one of the control arrangement or the server is further configured to:
receive a copy of the medical device data transmitted from the second medical device to the first medical device; and
cause the copy of the medical device data to be displayed on the user interface of the clinician device.

57. The control arrangement of claim 55, wherein the filter routine is configured to determine a physical or proximal distance between the first medical device and the plurality of medical devices by:
determining a network-level closest network access point, router, or bridge to the first medical device;
determining network distances or proximities from the network-level closest network access point, router, or bridge;
applying physical distance or proximity estimations based on the determined network distances or proximities; and
selecting a number of the other medical devices that have a closest estimated physical distance or proximity to the first medical device.

58. The control arrangement of claim 57, wherein the filter routine uses a data link layer to determine the network distances or proximities for creating the network map.

59. The control arrangement of claim 58, wherein the server is configured to turn the network map into an algebraic expression for applying the physical distance or proximity estimations.

60. The control arrangement of claim 53, wherein the filter routine is configured to determine a physical or proximal distance between the first medical device and the plurality of medical devices by:
determining a network-level closest network access point, router, or bridge to the first medical device;
determining network distances or proximities from the network-level closest network access point, router, or bridge;
applying physical distance or proximity estimations based on the determined network distances; and
selecting the other medical devices that have an estimated physical distance or proximity that is less than a threshold.

61. The control arrangement of claim 60, wherein the threshold corresponds to a size of a room or a half-room.

62. The control arrangement of claim 53, wherein the first medical device uses the medical device data from the second medical device by displaying the medical device data in conjunction with second medical device data generated by the first medical device.

63. The control arrangement of claim 53, wherein the first medical device uses the medical device data from the second medical device by incorporating the medical device data into an equation with second medical device data generated by the first medical device to determine a medical parameter.

64. The control arrangement of claim 53, wherein the identified other medical devices are displayed in conjunction with at least one of a device identifier, a medical device type, an identifier of a medical device data type, an associated patient name, or a room location of the respective medical device.

65. The control arrangement of claim 55, wherein at least one of the control arrangement or the server is further configured to authenticate each of the plurality of medical devices after the respective medical device is connected to the network.

66. The control arrangement of claim 53, wherein the control arrangement is configured to create a record indicative that the second medical device is transmitting the medical device data to the first medical device.

67. The control arrangement of claim 55, wherein at least one of the control arrangement or the server is configured to cause the second medical device to stop transmitting the medical device data to the first medical device after receiving at least one of (i) an indication of a disconnection of at least one of the first medical device or the second medical device from the network, (ii) an end of treatment being performed by the first medical device, or (iii) a command received from the user interface indicative that the transmission should end.

68. A clinician device for specifying medical device interoperability, the clinician device including:
a display screen;
a processor in communication with the display screen; and
a memory in communication with the processor, the memory storing instructions that define or specify an application, that when executed by the processor, cause the processor to:
transmit a query message to a server connected to a memory device storing a network map of a network that is indicative of connectivity between access points, network switches, routers, bridges, and a plurality of medical devices that are located within a medical facility, each of the medical devices including at least one of a network address or a MAC address, the query message:
including a request to identify possible other medical devices of the plurality of medical devices that are located in a same area as a first medical device, the first medical device configured to use medical device data from other medical devices, and
causing the server to perform a filter routine using the network map to identify which other medical devices of the plurality of medical devices are proximally close to each other,
receive a message from the server that is indicative of the identified other medical devices, cause the identified other medical devices to be displayed via a user interface on the display screen,
receive a selection of one of the displayed other medical devices, the selection corresponding to a second medical device, and
transmit a message to the server indicative of the selection, causing the server to enable the second medical device to transmit the medical device data to the first medical device by at least transmitting the at least one of the network address or the MAC address of the first medical device to the second medical device.

69. The clinician device of claim 68, wherein the memory stores additional instructions that define or specify the application, that when executed by the processor, cause the processor to:
receive the medical device data transmitted by the second medical device to the first medical device; and
display the received medical device data within the same user interface or a different user interface on the display screen.

70. The clinician device of claim 68, wherein the first medical device uses the medical device data from the second medical device by displaying the medical device data in conjunction with second medical device data generated by the first medical device.

71. The clinician device of claim 68, wherein the identified other medical devices are displayed in conjunction with at least one of a device identifier, a medical device type, an identifier of a medical device data type, an associated patient name, or a room location of the respective medical device.

72. A method to enable medical device interoperability, the method including:
storing in a memory device a network map of a network that is indicative of connectivity between access points, network switches, routers, bridges, and a plurality of medical devices that are located within a medical facility, each of the medical devices including at least one of a network address or a MAC address;
receiving, in a server that is connected to each of the plurality of medical devices via the network, a query message from a clinician device to identify possible other medical devices of the plurality of medical devices that are located in a same area as a first medical device, the first medical device configured to use medical device data from other medical devices;
performing, via the server, a filter routine using the network map to identify which other medical devices of the plurality of medical devices are within a close proximity of each other;
causing, via the server, the identified other medical devices to be displayed via a user interface of the clinician device;
receiving, in the server, a selection of one of the displayed other medical devices, the selection corresponding to a second medical device; and
enabling, via the server, the second medical device to transmit the medical device data to the first medical device by at least transmitting the at least one of the network address or the MAC address of the first medical device to the second medical device.
